(19) United States
(12) Patent Application Publication
(10) Pub. No.: US 2017/0363614 A1
(43) Pub. Date: Dec. 21, 2017

(54) Methods For Screening Therapeutic Compounds

(71) Applicant: Enumeral Biomedical Holdings, Inc., Cambridge, MA (US)

(72) Inventors: Arthur H. Tinkelenberg, Oradell, NJ (US); Anhco Nguyen, Needham, MA (US); Maria Isabel Chiu, Newton, MA (US); Aleksander Jonca, Boston, MA (US); Thomas McQuade, Cambridge, MA (US); Sri Sahitya Vadde, Burlington, MA (US); Sheila Ranganath, Arlington, MA (US)

(21) Appl. No.: 15/538,612

(22) PCT Filed: Dec. 19, 2015
(86) PCT No.: PCT/US2015/066955

§ 371 Date: Jun. 21, 2017

Related U.S. Application Data
(60) Provisional application No. 62/095,704, filed on Dec. 22, 2014.

(51) Int. Cl. G01N 33/50; G01N 33/68

(52) U.S. Cl. CPC G01N 33/5023; G01N 33/6854; G01N 33/5047; G01N 2333/70514; G01N 2333/54; G01N 2333/535; G01N 2333/70517; G01N 2333/70596; G01N 2333/57

(57) ABSTRACT

Methods of selecting test compounds on the basis of their cellular response profiles are disclosed. For a given test compound, a cellular response is determined by introducing into an array of individually addressable microwells a population of cells comprising a plurality of cell types and contacting the cells with the test compound. The cellular response profile for the test compound is then compared to a desired cellular response profile, and the test compound is selected based on the comparison.

This application claims the benefit of U.S. Provisional Application No. 62/095,704, filed on Dec. 22, 2014. The entire teachings of the above application are incorporated herein by reference.

### BACKGROUND OF THE INVENTION

In the search for new or improved therapeutic agents, the initial screening and in vitro characterization of candidate molecules, including small molecules and monoclonal antibodies, typically is performed by means of binding assays. Numerous methods and assays have been developed for detection of binding. Conventional in vitro screening typically is based on immunoassays such as ELISA, which have been adapted into automated multiplex assays that enable rapid screening of large numbers of molecules. After candidate molecules have been identified in initial screens, binding properties such as specificity, affinity, avidity, on-rate, off-rate, and the like can be analyzed using methods such as surface plasmon resonance. Nevertheless, even if they display excellent binding to the target in vitro, most drugs fail in subsequent in vivo testing or in clinical trials. Various factors other than the strength and specificity of the binding to the drug target can be responsible for such failure.

Biological response to candidate molecules (test compounds) can be at least as important as the quality of the binding interaction. A biological response (e.g., cellular response) to a test compound can be influenced by factors including: which different cell types display the target on the cell surface, the timing of the cell surface display, the density or abundance of the target on the cell surface, limitations on availability of the target (due to phenomena such as receptor internalization and/or degradation and cell membrane microdomain localization or other trafficking effects), and the presence or activation state of molecules downstream in signaling pathways connected to the target. Consequently, there is a need for a rapid, in vitro multiplex assay method that is suitable for characterizing test molecules, e.g., antibodies and small molecules, with respect to relevant biological function, e.g., cellular function, across various potentially relevant cell types.

### SUMMARY OF THE INVENTION

The current invention provides a rapid method for generating a cellular response profile for a test compound(s). Such cellular response profiles are useful for determining whether a test molecule elicits relevant cellular responses across a plurality of cell types. If the cellular response profile of the test molecule matches, or aligns satisfactorily with, a desired response profile, then the test molecule can be selected, such as for further study or drug development.

Accordingly, the present invention relates to a method of selecting a test compound having a desired cellular response profile. A desired cellular response profile is identified for a physiological condition of interest along with a profile comparison criterion that relates to the desired cellular response profile. For a test compound, a cellular response is then determined by introducing into an array of individually addressable microwells a population of cells comprising a plurality of cell types distinguishable by the presence or absence of cell surface markers; contacting the cells with the test compound in the absence of other test compounds; distinguishing cell types on a microwell-by-microwell basis; and detecting on a microwell-by-microwell basis the presence or absence of a cellular response by the cells to the test compound. The cellular response profile for the test compound is then compared to the desired cellular response profile to determine whether the profile comparison criterion is satisfied; and a test compound is selected that satisfies the profile comparison criterion.

These and other aspects and advantages of the invention will become apparent upon consideration of the following detailed description and claims. As used herein, “including” means without limitation, and all examples cited are non-limiting.

### BRIEF DESCRIPTION OF THE DRAWINGS

The foregoing will also be apparent from the following more particular description of example embodiments of the invention, as illustrated in the accompanying drawing.

FIG. 1 shows cellular response profiles for different test compounds.

### DETAILED DESCRIPTION OF THE INVENTION

In the methods disclosed herein, the test compounds are assayed for their ability to trigger one or more cellular responses in different cell types, in an in vitro multiplex assay. In contrast to conventional screening assays that assess binding of a test compound(s), in some embodiments, any of numerous potential cellular responses can be assayed for a plurality of different cell types.

In some embodiments, the present invention provides methods of selecting a test compound having a desired cellular response profile for a physiological condition of interest by determining a cellular response profile for a test compound, comparing the cellular response profile for the test compound to the desired cellular response profile to determine whether a profile comparison criterion is satisfied; and selecting the test compound for which the profile comparison criterion is satisfied.

A wide variety of test compounds can be used in connection with the present invention, including, for example, macromolecules (e.g., nucleic acids, proteins, antibodies, SURROBODY™ binding proteins, cytokines, and chemokines) and small molecules. In some embodiments of the invention, the test compound is an antibody, e.g., a monoclonal antibody. In some embodiments, the test compound is a compound of interest that needs to be characterized with respect to its effect, if any, on a cellular response that can be measured in microwells such as those described herein. In some embodiments, compounds are chosen for study, or test compounds are selected (e.g., for further characterization, for study as a lead candidate, or for drug development), based on a hypothesis (e.g., an a priori drug mechanism hypothesis), empirical model, or theory. In other embodiments, compounds are chosen for study at random. In some embodiments, compounds are chosen for study, or test compounds are selected, based on a hypothesis emerging from profiling across a patient cohort.

As used herein, “test compound” means: (a) a particular molecule; or (b) a particular combination of molecules.

As used herein, unless indicated otherwise, “antibody” means an intact antibody or antigen-binding fragment of an antibody, including an intact antibody or antigen-binding fragment that has been modified or engineered, or that is a human antibody. Examples of antibodies that have been modified or engineered are chimeric antibodies, humanized antibodies, multiparatopic antibodies (e.g., biparatopic antibodies), and multispecific antibodies (e.g., bispecific antibodies). Examples of antigen-binding fragments include Fab, Fab′, F(ab′)₂, Fv, single chain antibodies (e.g., scFv), minibodies and diabodies.

A “cellular response profile” for a test compound is a profile of cellular responses that are elicited by a given test compound across a plurality of cell types. The profile can be generated, for example, by aggregating or averaging cellular responses across some or all cells of a given cell type (or subtype) to generate an aggregated/average cellular response exhibited by that cell type in response to administration of the test compound. In some embodiments, the profile can further include a measure of the variance of the cellular response amongst the cells within a given cell type.

A “cellular response” is a response of a cell to contact with a test compound. A cellular response chosen for a particular embodiment of the invention will depend on the types of cells employed and the desired or expected physiological effect(s) of the test compound(s) on an organism, e.g., an animal or human, to which the test compound might be administered.

A “desired cellular response profile” is a cellular response profile that embodies, in whole or in part, a desirable response to a test compound. In some embodiments, the desirable cellular response profile is an ideal or optimal response profile. In other embodiments, the desirable cellular response profile is an acceptable, though not necessarily optimal, response profile. In some embodiments, the desired cellular response profile involves multiple factors, e.g., increased secretion of IFN-gamma and IL-2 by T cells. In some embodiments, the desired cellular response profile involves only one factor, e.g., increased secretion of IFN-gamma by T cells. A desired cellular response profile is not necessarily the biological effect ultimately sought. For example, increased secretion of IFN-gamma by T cells found among tumor infiltrating lymphocytes (TILs) might be the desired cellular response profile, while eradication of the tumor might be the biological effect ultimately sought.

Desired cellular response profiles can be based on a wide variety of factors, including, for example, theories, models, empirical data, or understandings by those of skill in the art regarding how various cellular responses by various cell types can trigger favorable and/or unfavorable physiological responses to test compounds. Those of skill in the art will recognize attributes of a test compound that represent a “desired” profile, such as a balance of predicted efficacy and safety. For example, in the context of drug screening and development, a desired cellular response profile for a drug could include the induction of cytokine secretion in one immune cell type, but not in another, if hypothetically, the induction of cytokine secretion in the first type of immune cell was understood to mediate a therapeutic response, while the induction of cytokine secretion in the second type of immune cell was understood to mediate no effect, or an undesired side effect. Such a desired cellular response profile could be based on an understanding about the mechanism of action of a test compound. In the context of comparing the activities of a test compound with a reference compound, the desired profile could simply be one that is obtained for a reference compound that is known to produce the biological effect ultimately sought.

A desired cellular response profile can include desired cellular response data for all cell types studied, for some cell types, or for one cell type. Further, a desired cellular response profile can include one or more desired cellular responses for one or more cell types. Optionally, the desired cellular response profile is established before cellular responses of the test compound are determined.

Methods of the present invention can be used in relation to a wide variety of physiological conditions, including normal and abnormal (e.g., disease) conditions. Examples of disease conditions include those mediated by infectious agents (bacteria, viruses, protozoa, fungi, prions, etc.), autoimmune diseases, metabolic diseases, diabetes, cancer, and inherited genetic conditions. Desired cellular response profiles can be used, for example, to select a test compound for use in further drug development for treating one or more abnormal physiological conditions.

A “profile comparison criterion” is one or more criteria used to determine whether the comparison of a cellular response profile for a test compound with a desired cellular response profile results in the selection of the test compound. A wide variety of profile comparison criteria can be used, such as, for example, requiring an exact match between the cellular response profile for a test compound and the desired cellular response profile in order for the test compound to be selected. In some embodiments, the profile comparison criterion is based on a comparison of one, two, or any number of responses in the cellular response profile with corresponding responses in the desired cellular response profile.

In some embodiments, the profile comparison is that all cellular responses in the cellular response profile for a test compound are identical to the corresponding responses in the desired response profile. In some embodiments, the profile comparison criterion is whether there is an acceptable level of similarity between some or all of corresponding responses in the two profiles. For example, the profile comparison criterion might be no more than a 5%, 10%, 25%, or 50% difference between one or more corresponding cellular responses. In some embodiments, the profile comparison criterion is simply a significant difference in the parameter being measured. Some profile comparison criteria could involve weighing the level of agreement between some corresponding cellular responses as relatively more important to the decision-maker than the level of agreement between other corresponding cellular responses. In some embodiments, the profile comparison criterion could require a lack of agreement between at least some cellular responses in the cellular response profile of the test compound and corresponding responses in the desired cellular response profile.

In some embodiments of the invention, the profile comparison criterion could be the satisfaction of any of several alternate selection conditions. In this manner, a test compound can be selected, for example, if it demonstrates a certain combination of profile properties believed to render it a promising lead drug candidate in patient subpopulation A, without necessarily requiring that it also be promising in subpopulation B, or optimal across the population as a whole. The invention can be readily adapted by those of skill in the art to address challenges in the field of personalized medicine, such as, for example, delivering medicines that are effective in different subpopulations of patients suffering from a condition. For example, a drug that is effective to treat Patient A could be predicted to be ineffective in Patient B, for example, due to other disorders Patient B has, or medications Patient B is taking, and vice versa. Methods of the invention can be used to probe manifestations of these differences, such as cellular response heterogeneity, and thereby to identify test compounds that are predicted to be effective in different individual patients, given the determined cellular response profiles for those test compounds.

Returning to examples of various cellular responses that can be determined in embodiments of the present invention, it is noted that cellular responses can be the secretion of a molecule, such as a cytokine, chemokine, growth factor, hormone, or neurotransmitter, by a cell. Examples of cytokines useful in the invention include interferon (IFN)-gamma, interleukins, e.g., IL-1, IL-2, IL-4, IL-5, IL-6, IL-9, IL-10, IL-12, IL-13, IL-15, IL-17 IL-18, IL-19, IL-22, IL-23, IL-25, IL-33, thymic stromal lymphopoietin (TSLP), glycosylation-inhibiting factor (GIF), Mast Cell Activation-Related Chemokine (MARC), LTC4, PGD2, Granzyme B, tumor necrosis factor alpha (TNFα), and Granulocyte-macrophage colony-stimulating factor (GM-CSF).

Additional examples of cellular responses include the following:
a) production and/or secretion of an antibody;
b) intracellular production of a substance, e.g., cytokine, chemokine, growth factor, hormone, neurotransmitter, second messenger, transcription factors, factors, cytokine;
c) changes in cell morphology;
d) changes in cell motility;
e) changes in rate of cell movement;
f) changes in distance traveled by cells within the microwells;
g) gene activation;
h) transcriptional changes;
i) change in activation state of one or more receptors;
j) conformational changes of one or more proteins, within the cell membrane and/or within the cytoplasm or specific organelles;
k) change in cell surface topology, such as changes in the identity, number, or density of one or more cell surface proteins (including cell markers);
l) changes in cell division or differentiation;
m) apoptosis;
n) toxicity or cell death (e.g., necrosis);
o) change in membrane permeability;
p) up or down regulation of genes;
q) changes in cell metabolism; and
r) uptake of a test compound by the cell.
   Without desiring to be held to any particular mechanism of action, it is noted that those skilled in the art will appreciate a variety of mechanisms by which a test compound can interact with a cell to induce a cellular response. One mechanism is signal transduction, wherein a test compound activates a specific receptor located on the cell surface or inside a cell, the receptor in turn triggering a biochemical chain of events, creating a cellular response. For example, a test compound could associate with the extracellular domain of an integral transmembrane protein membrane protein (e.g., G protein-coupled receptor), inducing a conformational change in the protein that transduces a signal across the cell membrane, resulting in the activation of an enzyme in the receptor or the exposure of a binding site for other intracellular signaling proteins within the cell. Other extracellular receptors that can mediate cellular response following binding by a test compound include tyrosine and histidine kinases, integrins, toll-like receptors, and ligand-gated ion channels. A number of intracellular receptors can also mediate cellular responses by a test compound, including nuclear receptors and cytoplasmic receptors.

The binding of a test compound with a target is not considered per se to be a “cellular response.” Notwithstanding, it should be appreciated that methods of the present invention can be extended or adapted for applications wherein binding of a test compound is measured or observed. In a manner analogous to the determination of a cellular response profile, a cellular binding profile could be determined for a test compound across a plurality of cell types in a cell population. Determinants of binding of a test compound could include, for example, the affinity, specificity, avidity, on-rate, and off-rate with which the test compound binds to a receptor or target on a cell. A desired binding profile could also be established, along with a binding profile comparison criterion. The determined and desired cellular binding profiles could be compared to determine whether the binding profile comparison criterion is satisfied, and if so, the test compound could be selected. Further, it should be appreciated that extended cellular profiles and extended desired profiles can be constructed that include both cellular response and binding response data. Extended profile comparison criteria (e.g., criteria based on a comparison of both cellular binding and cellular response) can be adapted to facilitate comparisons in which binding and cellular response data are considered.

In some embodiments, immune cells are used as test cells in research to identify pharmacologically useful immuno-modulators, and secretion of a cytokine can be a specific response measured in the assay. In some embodiments, the test cells are immune cells such as CD4+ cells, CD8+ cells, T-regulatory cells, NK cells, macrophages or innate lymphoid cells (ILCs); and IFN-γ secretion is the cellular response measured.

The measurement or characterization of cellular responses can be achieved in a wide variety of ways, as will be apparent to those of skill in the art. Cellular responses can be measured qualitatively (e.g., presence or absence of a certain response) or quantitatively (e.g., measurement or determination of concentration of produced/secreted cytokine). Cellular responses can also be reported in a variety of ways, including as raw data, normalized data, and processed data. One form of processing could be, for example, a comparison of baseline data (data in the absence of a test compound) and the raw data, e.g., a subtraction of baseline data or raw data. For example, a cellular response could be a change in the density of a particular surface protein following administration of a test compound, calculated as the surface protein density in the presence of test compound minus the surface protein density in the absence of test compound.

For some embodiments, cellular response(s) can be determined by examining a property of a solution in which the cell resides, e.g., the presence or concentration of a cytokine that is secreted by the cell in response to the test compound. Determination of other cellular responses can require examination of the cells themselves, e.g., to assess whether a particular marker is expressed on the outside of a cell in response to contact with the test compound. In some embodiments, the determination of a cellular response involves destruction of a cell, e.g., lysis followed by analysis of cell contents, such as an antibody that is produced (but not necessarily secreted) in response to contact with test compound. In some embodiments, the cellular response measured is an intracellular (non-secreted) molecule that has been liberated from an intact cell, i.e., released into the surrounding medium, e.g., by a cell lysis or permeabilization step incorporated into an assay protocol. In some embodiments, the determination of cellular response(s) does not involve destruction of the cell, which can permit the same array of cells to be studied (e.g., interrogated) multiple times with different test compounds.

Without wishing to be bound by theory, it is believed that the response of a cell to a test compound can be influenced by the cell surface microenvironment surrounding a molecule that is targeted by the test compound (e.g., that the test compound binds to), and that the cell surface microenvironment surrounding a target molecule will vary by cell type. Therefore, in some embodiments, a test compound, or plurality of test compounds having the same target (e.g., cytokine receptors or immunomodulatory receptors) will elicit different cellular responses from different cell types expressing that target. ERBB2 and ERBB3 provide an additional example of this. See Xiaolan Qian et al., Proceedings of the National Academy of Sciences USA, vol. 91, pp. 1500-1504 (February 1994).

In some embodiments, the determination of a cellular response will involve a microengraving process as more fully described in U.S. Pat. No. 8,835,187.

In some embodiments the cellular response is one that can be detected on equipment that is suitable to automation, and preferably high throughput screening. A variety of optical methods are well known in the art to rapidly characterize arrays of cells or other materials. In some embodiments, an enzyme-linked immunosorbent assay (ELISA) is used to determine the presence or absence (within limits of detection) of a secreted protein in response to contact by a test compound. In some ELISA assays, for example, antigens from a sample are attached to a surface, and an antigen-specific antibody is applied over the surface and binds to the antigen. The antigen-specific antibody is linked to an enzyme. Addition of the enzyme's substrate produces a detectable signal, e.g., a color change in the substrate. In some embodiments the cellular response is characterized by a fluorescence signal (e.g, by using fluorescently-labeled antibodies to a secreted protein). In some embodiments, multiple cellular responses are detected at the same time, e.g., by using appropriate detection reagents, specific to each of the cellular responses, and providing a non-overlapping signal (e.g., a fluorescence signal).

In some embodiments, cellular responses are characterized as the presence or absence of a cellular response, such as the presence or absence of detectable antibody or cytokine secretion. The “presence” of a cellular response can be defined in a variety of ways, such as a measured response being greater than a certain numerical value, or as the presence of an observable response (e.g., a response above a limit of detection).

In some embodiments, the cellular response profile for a test compound is determined by (a) introducing into an array of individually addressable microwells a population of cells comprising a plurality of cell types identifiable by the presence or absence of cell surface markers; (b) contacting the cells with a detectable agent that specifically binds a cell surface marker; (c) contacting the cells with the test compound in the absence of other test compounds; and detecting, on a microwell-by-microwell basis, (i) the presence or absence of the cell surface markers; and (ii) the presence or absence of a cellular response by the cells to the test compound; (d) comparing the cellular response profile for the test compound to the desired cellular response profile; and (e) selecting the test compound having the desired cellular response profile.

A variety of cell populations can be used in connection with methods of the present invention. Cell populations may derive, for example, from an individual subject or a plurality of subjects, from normal tissue or diseased tissue, and from tissue that is excised from subjects or grown in vitro. Cell populations include populations of cells sharing one or more defining characteristics, such as a common origin, classification, morphology, or feature. Examples of cell populations include a human cell population, an immune cell population, a cancer cell population, an animal cell population, a hydridoma cell population, a population of cells undergoing apoptosis, a population of cells obtained by dissociation of a tumor tissue sample, and so on.

As used herein, “a cell type” is a type of cell within the cell population. As such, a cell type has at least one feature or characteristic that serves to differentiate it from other cell types in the cell population. A wide variety of distinguishing features or characteristics can be employed, including features that are structural, functional, or both. Cell types include, but are not limited to, immune cells within a broader population of human cells. Cell types can be defined by the presence or absence of one or more surface markers on otherwise identical cells. Phenotypically distinguishable cells within a given category of cells can be different cell types. Within a population of immune cells, cell types could include, for example, T cells and B cells. Further, within a cell population of T cells, cell types could include T lymphocytes, T helper cells, and T regulatory cells. In the alternative, within a population of immune cells, cell types could be considered to be T lymphocytes, T helper cells, T regulatory cells (Treg), and B cells.

In some embodiments, the cell types include different types of immune cells. In some embodiments, the immune cells are selected from the group consisting of CD4+ cells, CD8+ cells, Treg cells, natural killer (NK) cells, macrophages, innate lymphoid cells (ILCs) and myeloid-derived suppressor cells (MDSCs). In some embodiments, cell populations and cell types can include epithelial cells, endothelial cells, hormonal cells, neuronal cells, cardiac cells, kidney cells, liver cells, stem cells, tumor cells, mesenchymal cells, and cell types in transition, such as those undergoing epithelial-mesenchymal transition (EMT).

In some embodiments, cell types are distinguishable from one or more other cell types by the presence or absence of cell surface markers, either alone or in combination. Further, in some embodiments, the cell surface markers are sufficient to identify one or more cell types. A wide variety of surface markers can be used in connection with the present invention. Appropriate surface markers can be readily identified by those of skill in the art. For example, those of skill in the art will recognize that a population of immune cells can be probed by the application of cluster of differentiation (CD) protocols for immuno-phenotyping of cells. Because T cells can differ in their expression of specific cell surface markers, for example, CD8+ T cells and CD4+ T cells can be treated as different cell types for purposes of the present invention. Similarly, any two cells that differ with respect to at least one phenotypic marker can be considered different cell types, depending on the context, e.g., the assay design in which they are used. It should be understood that methods of identifying cell types other than via cell surface markers can be used. The selection of suitable methods for identifying particular cell types can be made by those of skill in the art.

Cells in the cell population can be deposited in or on a variety of structures, substrates, containers, plates, or arrays in accordance with methods of the present invention. In some embodiments, cells in the cell population are introduced into an array of microwells (also known as “nanowells” in some publications).

Various microwell devices can be used. In some embodiments, the microwells are in the form of a two-dimensional array. In some embodiments, the microwells are approximately 50-100 microns in diameter and approximately 50-100 microns deep. In some embodiments, the dimensions of each microwell are approximately 50 microns×50 microns×50 microns, resulting in a well volume of 125 picoliters. Microwells of approximately 125 pL or less are suitable for containing a single cell or a few cells, e.g., 1-5 cells in a volume small enough to minimize dilution of cellular excretion products, thereby lowering the limits of detection of the products.

In some embodiments, less than about 5 cells be deposited in each microwell, for example, about 1 cell per microwell. In some embodiments, cellular responses from cells occupied by more than a given number of cells, e.g., more than one cell, are ignored for purposes of data analysis.

In some embodiments, the microarray is fabricated from a flexible or compliant material. For example, poly(dimethylsiloxane) (PDMS), an elastomeric material, is used for this purpose, because it is biocompatible and gas permeable, as well as flexible. In some embodiments, a micro array is formed from a rectangle of PDMS that is approximately 1 mm thick and adhered to a conventional 3 inch×1 inch glass microscope slide. Flexibility or compliance of the material facilitates formation of a tight, but reversible, seal on top of the microwells, when the array is compressed against a smooth, rigid substrate such as a glass microscope slide. When sealed in this way, the microarray and the associated rigid substrate together can constitute a “device” or “microarray device” suitable for use in the methods disclosed herein. The microwells that constitute the microarray can be formed by photolithography carried out on a microarray-forming substrate such as a thin (approximately 1 mm) slab of PDMS. In some embodiments, the addressable (ordered) microwells are arrayed in one or more rectangular patterns containing a total of at least 1,000 microwells in the device, e.g., at least 20,000 microwells, e.g., at least 50,000 microwells. In some embodiments, the array contains approximately 85,000 microwells, i.e., 84,672 microwells arranged in a rectangular 24×72 grid of smaller 7×7 grids. Suitable microarrays and methods of making and loading them with cells have been described. See, e.g., Love et al., U.S. Pat. Nos. 7,776,553; 8,865,479; 8,835,187; and U.S. Pat. No. 8,772,049; Varadarajan et al., 2012, Proc. Nat'l Acad. Sci. USA 109:3885-3890 and U.S. Patent Publication No. US20120015824.

In some embodiments these microwells are “individually addressable.” “Individually addressable” means that cellular responses can be individually determined for each microwell, and that cellular response data for a cell can be associated with cell surface marker data for the same cell. Various schemes for individually addressing locations within microarrays are known in the art, and can include, for example, various methods of indexing and spatial registration of instrumentation.

In some embodiments, detectable agents are used to detect a cell surface marker. A variety of such agents can be used, including antibodies that bind to the cell surface marker. Cells can be contacted with these detectable agents according to methods known in the art. For example, anti-CD4 antibodies can be used to detect the presence of CD4 surface markers on immune cells. Various properties can render an agent “detectable,” including, for example, optical properties (e.g., fluorescence), nuclear properties (e.g., spin of labelled atoms, radioactivity of labelled atoms), magnetic properties (e.g., magnetic beads, coupled to the agent). Some detectable agents are labelled reagents, such as macromolecules comprising a first region responsible for binding (including specific binding) to a cell surface marker, and a second region responsible for detectability (e.g., an appended fluorescent moiety).

Modification of antibodies for use as components of detectable agents is well known in the art. For example, antibodies may be modified with a ligand group such as biotin, or a detectable marker group such as a fluorescent group, a radioisotope, or an enzyme. Antibodies of the invention can be labeled using conventional techniques. Suitable detectable labels include: fluorophores, chromophores, radioactive atoms, electron-dense reagents, enzymes, and ligands having specific binding partners. Enzymes are typically detected by their reaction products. For example, horseradish peroxidase can be detected through conversion of tetramethylbenzidine (TMB) to a blue pigment, quantifiable with a spectrophotometer. For detection, suitable binding partners include biotin and avidin or streptavidin, IgG and protein A, and numerous receptor-ligand couples known in the art. Other permutations and possibilities will be readily apparent to those of ordinary skill in the art.

The step of contacting the cells with the test compound can be performed in a variety of ways, including exposing the cell to test compound and delivering the test compound intracellularly. Optionally, the test compound is administered after the cell has been primed with one or more other compounds. The priming step can involve, for example, the permeabilizaton of the cell membrane or activation of the cell to enhance or modulate the response to achieved by the contact of the cell with the test compound. For example, cells can be stimulated with CD3/28 and then treated with PD-L1. Test compound anti-PD-1 antibodies can then be added to the wells. After three days, the samples can be microengraved, and interferon gamma (IFNγ) secretion assessed, and the percentage of IFNγ-secreting wells can be calculated. The ability of the test anti-PD-1 antibodies to block the inhibitory effect of PD-L1, thus decreasing PD-1/PD-L1-mediated inhibition, can then be assessed. In some embodiments, the contacting step can be performed in a batch manner on the entire population of the cells. In some embodiments, the contacting step is performed on cells that have been previously deposited into microwells (e.g., before the cells are introduced into an array). In some embodiments, the contacting step is performed after cells have been introduced into an array.

In some embodiments, the cellular response is measured within 24 hours after the test compound contacts the cells, e.g, it is measured within approximately 6 hours. In some embodiments, the cellular response is measured in approximately 2 hours, and in some embodiments, the cellular response is measured within minutes or even seconds. The length of time between cellular contact with the test compound and measurement of the cellular response (“incubation period”) will depend on the particular cellular response being measured. Determination of a suitable incubation period for different embodiments of the invention is within ordinary skill in the art.

In some embodiments, each test compound can be administered in the absence of other test compounds. Notwithstanding, it should be appreciated that two or more test compounds can be administered together, and the response measured as described herein. In other embodiments, a substance, compound, adjuvant, substrate, or primer molecules can be co-administered to the population of cells (or a subpopulation thereof) along with each test compound, e.g., molecule.

The cellular response profile for the test compound can then be compared to the desired cellular response profile to determine whether the profile comparison criterion is satisfied. The comparison step can be performed in a wide variety of ways. In some embodiments, all cellular responses within the cellular response profile are involved in the comparison. In some embodiments, only subsets of cellular responses are compared.

If the profile comparison criterion is satisfied, then the test compound is selected. The selected compound can optionally be used for further study, analysis, experimentation, or development. For example, as part of a drug screening or development process, a selected test compound could serve as a starting point for further lead generation and refinement. As another example, a selected test compound could be advanced to a next stage of drug development, e.g., pre-clinical trials or Phase I clinical trials. In the alternative, the selection of the test compound is not followed by further actions. For example, in an experiment to validate that a test compound is equivalent to a reference compound, the cellular response profile of a reference compound could be considered to be the desired cellular response profile and the cellular response profile of the test compound compared therewith. If the comparison shows the response to be equivalent, then the test compound is selected as an equivalent compound to the reference compound. In this example, the selection step is essentially a validation of equivalence.

### EXAMPLES

The following Examples are merely illustrative, and are not intended to limit the scope or content of the invention in any way.

### Example 1

A desired cellular response profile for Disease X is determined to be as follows: In response to contact with a test compound, immune cells of Type A secrete cytokine Y and immune cells of Type B do not secrete cytokine Y. According to an understanding of the pathophysiology of Disease X, induction of cytokine Y secretion in Type A immune cells could mediate a beneficial therapeutic effect; while induction of cytokine secretion in Type B immune cells could cause an undesirable side effect.

For detection of cytokine, affinity-purified antibodies that bind to the cytokine are labeled by conjugating the antibodies with NETS-ester activated fluorescent dyes, and purified by spin column. Alternatively, biotinylated antibodies against cytokine Y and fluorescent streptavidin are used. A population of immune cells comprising Type A and Type B cells is introduced into an array of 1024 microwells (0.1-1 nL each), fabricated by a combination of photolithography and replica molding of monolithic slabs of poly(dimethylsiloxane) (PDMS). Suspensions of immune cells are deposited on the surface of the PDMS containing the microwells. Visual inspection of the slabs by microscopy confirms that the wells contain approximately 1 to 2 cells/well with a loading efficiency of 50-70%.

Test Compound TC1 is introduced into each of the microwells and allowed to contact the cells for approximately 6 hours. To detect secreted cytokine Y, the array of microwells loaded with cells is placed face down on a glass slide that has been prepared by immobilizing the antibodies against cytokine Y on the glass slide (via functionalized epoxide-bearing silanes), rinsing the slide with phosphate buffered saline (PBS) and blocking with bovine serum albumin.

This configuration confines the cells to discrete, closed compartments with volumes of about 0.1 nL each. The device is held together under light compression and incubated for 1 hour at 37° C. After the incubation, the array of microwells is removed and the glass slide placed into a blocking buffer. The glass slides bearing the captured antibodies and cytokines are contacted (e.g., interrogated) with anti-cytokine Y antibodies conjugated to fluorescent dyes. After blocking, the glass slides are dried by centrifugation. Appropriate detection agents are applied. After incubation, the slides are washed and spun dry. Images of the microarrays are collected on a laser-based microarray scanner and analyzed using the accompanying software.

To determine cell type, cells are stained with detectable-labeled anti-CD4 and anti-CD8 antibodies. Cell Type A is CD4+/CD8- and Cell Type B is CD4−/CD8+. Detection is performed by visual inspection under a microscope.

Of the 1024 microwells, 800 microwells are found to contain only one cell per well. Data from the remaining microwells are discarded. Of the 800 microwells containing only one cell per well, 350 microwells are found to contain Type A immune cells, 250 microwells are found to contain Type B immune cells, and 200 microwells are found to contain other immune cell types by absence of expression of CD4 and CD8 (CD4−/CD8−). TC1 produces a detectable secretion of cytokine Yin 300 of the 350 Type A immune cells (about 86%) and in only 10 of the 250 Type B immune cells (about 4%). TC1 also produces cytokine Y secretion in 100 of the 200 other/uncharacterized cell type microwells (50%).

The cellular response profile of TC1 (secretion of cytokine Y in 86% of Type A cells and 4% of Type B cells) is compared to the desired cellular response profile (100% secretion in Type A and 0% in Type B cells). The response profile of TC1 is deemed to be acceptably similar to the desired profile, and TC1 is selected for further study.

### Example 2

Example 2 is similar to Example 1, but the response profile for TC1 is not deemed to be acceptably similar to the desired profile, because 4% is an unacceptably high rate of secretion inducement in Type B cells. It is feared that this could lead to side effects presenting a serious safety issue. Therefore, TC1 is not selected. Sequentially or simultaneously, TC2 is tested on a second array of cells, using parameters otherwise similar to the assay for TC1. TC2 induces secretion of cytokine Yin 66% of Type A cells and in 0.5% of Type B cells. The response profile of TC2 is deemed to compare favorably to the desired response profile, and TC2 is selected for further drug development.

### Example 3

Additional detection agents are used that bind to the cell surface markers of the cells in the cell population of Example 1. This allows for the definitive determination of two subtypes of Type A immune cells, Type A1 and Type A2, having a relative abundance in the cell population of about 86% and 14%, respectively. It is further discovered that there are three variants of Type B immune cells, B1, B2 and B3, having a relative abundance of 85%, 10%, and 5%, respectively.

It is determined that TC1 induces secretion of cytokine Yin almost 100% of A1 cells, 0% of A2 cells, <1% B1 cells, <1% B2 cells, and in about approximately 80% of B3 cells. The desired response profile is refined to reflect an understanding that Type A1 cells are believed to be involved in mediating therapeutic responses, while Type A2 and A3 cells are not believed to be involved; and further that Type B1 cells are specifically implicated in side effects. The response profile of TC1 compares favorably to the refined desired response profile, because TC1 produces a secretion response in all of the Type A cells believed to mediate a therapeutic response (Type A1), and it produces virtually no secretion response in the Type B cells believed to mediate side effects (Type B1). TC1 is selected for further drug development. It is noted that TC2 will necessarily compare less favorably, because the maximum Type A1 response is predicted to be less than for TC1 (see Example 2, showing 66% response for Type A, and assuming 85% abundance of Type A1 subtype). Further, TC2 shows 0.5% binding to Type B cells, which is deemed not to be an advantage over TC1, which shows 4% binding overall to Type B cells but <1% binding to B1 subtype, which is believed to mediate side effects. Accordingly, TC1 is selected for further drug development in preference to TC1. No further studies are performed on TC2.

### Example 4

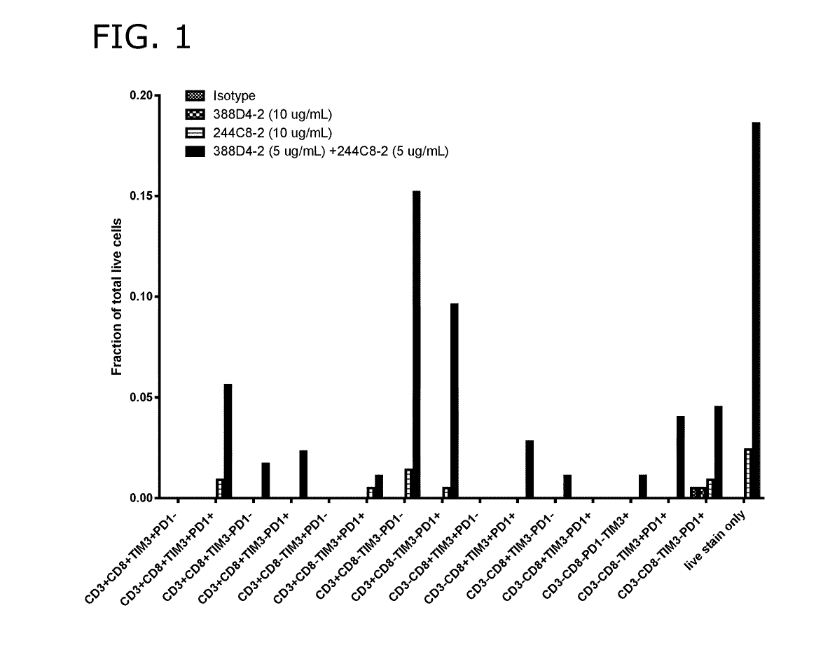

As shown with reference to FIG. 1, an experiment was performed to measure the increase in human T cell effector function, as indicated by IFNγ secretion by human TILs (tumor-infiltrating lymphocytes), in response to PD-1 blockade with different test compounds. The first test compound was anti-PD-1 antibody 388D4-2 (which competitively inhibits binding of PD-L1 to PD-1). The second test compound was anti-PD-1 antibody 244C8-2 (which does not inhibit binding of PD-L1 or PD-L2 to PD-1). The third test compound was a combination of antibodies 388D4-2 and 244C8-2. A negative control consisting of an isotype (IgG4) immunoglobulin was included. Antibody 388D4-2 was a humanized variant derived from a murine anti-human PD-1 antibody designated 388D4, and antibody 244C8-2 was a humanized variant derived from a murine anti-human PD-1 antibody designated 244C8. The experiment was performed essentially as follows.

Higher IFNγ secretion by cells of interest (T cells identified as CD3+CD8+ and CD3+CD8−) was identified as the desired cellular response profile. The profile comparison criterion was identified as IFNγ secretion of greater than 0.05 (as fraction of total live cells expressing) by at least one cell type of interest.

A cellular response profile for each test compound was determined substantially as follows:

Fresh tumor samples from NSCLC (non-small cell lung cancer) patients who had undergone surgical resection of tumors were obtained from the Cooperative Human Tissue Network, National Cancer Institute. Analysis was performed using single-cell suspensions of tumor cells from these tumor samples. Solid tumor biopsy samples were mechanically disrupted into single-cell suspensions using a gentleMACS Dissociator (Miltenyi Biotec) with enzymes A, H and R.

A population of cells comprising a plurality of cell types distinguishable by the presence or absence of cell surface markers was introduced into an array of individually addressable microwells substantially as follows:

To achieve polyclonal stimulation of TILs that were among the dissociated tumor cells, a 96-well assay plate was coated with 0.5 μg/mL anti-CD3 (OKT3) in coupling buffer, overnight at 4° C. The antibody coating solution was removed, and the plate was washed. Tumor suspensions were re-suspended to a density of approximately 1.5×10⁶ cells per mL. Then 200 μL of this second suspension was added to each experimental well, together with 2 μg/mL anti-CD28 (clone 28.2, eBioscience). Cells were distinguishable by the presence or absence of cell surface markers (see below).

Cells were contacted with the test compound in the absence of other test compounds substantially as follows:

A population of 3×10⁵ cells, which included 17% lymphocytes (stimulated as described above) was incubated for 24 hours with antibody 388D4-2 (10 μg/mL), antibody 244C8-2 (10 μg/mL), or a combination of antibodies 388D4-2 and 244C8-2 (total antibody concentration 10 μg/mL).

Detecting on a microwell-by-microwell basis the presence or absence of a cellular response by the cells to the test compound: Following PD-1 blockade, cells and supernatants were collected for analysis by microengraving in a microwell array device, with the device and the method essentially as follows.

A glass slide-mounted PDMS slab measuring approximately 22×63×1 mm, and containing a microwell array of 84,672 cube-shaped microwells measuring 50 μM per side (the “microwell device”), that was prepared as described in U.S. Pat. Nos. 8,569,046 and 8,835,188, was loaded with liquid growth medium and approximately 200,000 of the cells that were prepared as described above. A glass capture slide bearing an immobilized antibody against IFNγ was gently clamped on the loaded microwell device, and the clamped device was incubated for one and one half hours, to allow time for capture of secreted IFNγ on the capture slide. Following this incubation, the capture slide was removed, processed with a secondary antibody for fluorescence visualization, and scanned with a microarray slide reader (GenePix®, Molecular Devices, Sunnyvale, Calif.).

Cell types were distinguished on a microwell-by-microwell basis substantially as follows:

The uncovered, loaded microwell device was gently washed by immersion in divalent cation-free PBS, and treated with live cell stain (calcein violet). The microwell device was gently washed again by immersion in divalent cation-free PBS, and then treated with primary antibodies against the cell surface proteins, CD3, CD28, PD-1, and TIM3. After processing for multiplex, fluorescence visualization of the cell surface proteins with secondary antibodies, the microwell array was imaged using a suitably equipped microscope (ImageXpress® Micro XLS, Molecular Devices, Sunnyvale, Calif.), programmed to distinguish microwells containing only a single cell.

In this experiment, it was found that, among the cells of interest, i.e., T cells (CD3+CD8+ and CD3+CD8−), treatment with the combination of antibodies evoked an IFNγ response in a larger number of individually examined cells than did treatment with either antibody alone.

These results obtained in this example indicated that the combination of a ligand-competitive, PD-1 antagonist antibody and a PD-1 ligand non-competitive, antagonist antibody increased the number of tumor-infiltrating T cells displaying an effector response, relative to treatment with either antibody alone. This result was observed in spite of the fact that total antibody concentration was held constant, and both antibodies were directed against the same target. Because the measurements in this experiment were carried out on individual cells, and included information on specific cell surface markers, as well as a cellular response, these measurements enabled the responses of phenotypically different cells to the test compounds to be compared. The results obtained in this example enabled selection of the combination of two particular anti-PD-1 antibodies for further evaluation.

### INCORPORATION BY REFERENCE

The relevant portions of teachings of all patents, published applications and references cited herein are incorporated by reference in their entirety.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A method of selecting a test compound, comprising the steps of:
(a) identifying a desired cellular response profile and a profile comparison criterion for a physiological condition of interest;
(b) determining a cellular response profile for the test compound by:
1. introducing into an array of individually addressable microwells a population of cells comprising a plurality of cell types distinguishable by the presence or absence of cell surface markers;
2. contacting the cells with the test compound in the absence of other test compounds;
3. distinguishing cell types on a microwell-by-microwell basis; and
4. detecting on a microwell-by-microwell basis the presence or absence of a cellular response by the cells to the test compound;
(c) comparing the cellular response profile for the test compound to the desired cellular response profile to determine whether the profile comparison criterion is satisfied; and
(d) selecting the test compound if the profile comparison criterion is satisfied.

2. The method of claim 1, further comprising the step of contacting the cells with a detectable agent that specifically binds a cell surface marker, wherein distinguishing cell types on a microwell-by-microwell basis comprises detecting the presence or absence of the cell surface markers.

3. The method of claim 2, wherein the detectable agent that specifically binds a cell surface marker is selected from the group consisting of an anti-CD3 antibody, an anti-CD4 antibody, an anti-CD8 antibody, an anti-CD78 antibody, and an anti-CD68 antibody.

4. The method of claim 1, wherein the cell types are selected from the group consisting of: different types of immune cells, different types of epithelial cells, different types of endothelial cells, different types of hormonal cells, different types of neuronal cells, different types of cardiac cells, different types of kidney cells, different types of liver cells, different types of stem cells, and different types of tumor cells.

5. The method of claim 1, wherein the plurality of cell types includes different types of immune cells.

6. The method of claim 5, wherein the immune cells are selected from the group consisting of CD4+ cells, CD8+ cells, Treg cells, NK cells, macrophages, ILCs and MDCSs.

7. The method of claim 1, wherein cellular response profiles are determined for a plurality of test compounds.

8. The method of claim 1, wherein the cells are contacted with the test compound before the cells are introduced into the array.

9. The method of claim 1, wherein the cells are contacted with the test compound after the cells are introduced into the array.

10. The method of claim 1, wherein the cellular response by the cells is secretion of a cytokine.

11. The method of claim 10, wherein the cytokine is selected from the group consisting of IFN-gamma, IL-1, IL-2, IL-4, IL-5, IL-6, IL-9, IL-10, IL-12, IL-13, IL-15, IL-17 IL-18, IL-19, IL-22, IL-23, IL-25, IL-33, thymic stromal lymphopoietin (TSLP), glycosylation-inhibiting factor (GIF), Mast Cell Activation-Related Chemokine (MARC), LTC4, PGD2, Granzyme B, TNFα, and Granulocyte-macrophage colony-stimulating factor (GM-CSF).

12. The method of claim 1, wherein each microwell in the array contains a volume of approximately 125 picoliters or less.

13. The method of claim 1, wherein the array contains at least 1,000 microwells.

14. The method of claim 1, wherein the test compound is a drug candidate.

15. The method of claim 14, wherein the drug candidate is a monoclonal antibody.

16. The method of claim 14, wherein the drug candidate is a small molecule.

17. The method of claim 1, wherein the test compound is a particular combination of molecules.

18. The method of claim 17, wherein the particular combination of molecules is (a) an anti-PD-1 antibody that competitively inhibits binding of PD-L1 to PD-1 and (b) an anti-PD-1 antibody that does not inhibit binding of PD-L1 to PD-1 and does not inhibit binding of PD-L2 to PD-1.

19. A method of selecting a test compound, comprising the steps of:
(a) identifying a desired cellular response profile and a profile comparison criterion for a physiological condition of interest;
(b) determining a cellular response profile for the test compound by:
1. introducing into an array of individually addressable microwells a population of cells comprising a plurality of cell types distinguishable by the presence or absence of cell surface markers;
2. contacting the cells with a detectable agent that specifically binds a cell surface marker;
3. contacting the cells with the test compound in the absence of other test compounds; and
4. detecting, on a microwell-by-microwell basis, (i) the presence or absence of the cell surface markers; and (ii) the presence or absence of a cellular response by the cells to the test compound;
(c) comparing the cellular response profile for the test compound to the desired cellular response profile to determine whether the profile comparison criterion is satisfied; and
(d) selecting the test compound if the profile comparison criterion is satisfied.
